(19) United States
(12) Patent Application Publication
(10) Pub. No.: US 2012/0016208 A1
(43) Pub. Date: Jan. 19, 2012

(54) METHOD AND SYSTEM FOR SELECTING ITEMS USING PHYSIOLOGICAL PARAMETERS

(75) Inventors: Joris Hendrik Janssen, Eindhoven (NL); Joanne Westerink, Eindhoven (NL); Egidius Leon Van Den Broek, Wien (AT)

(73) Assignee: Koninklijke Philips Electronics N.V., Eindhoven (NL)

(21) Appl. No.: 13/258,498

(22) PCT Filed: Mar. 29, 2010
(86) PCT No.: PCT/IB2010/051359

§ 371 Date: Sep. 22, 2011

(30) Foreign Application Priority Data
Apr. 2, 2009 (EP) 09157173.7

(51) Int. Cl. A61B 5/00

(52) U.S. Cl. 600/300

(57) ABSTRACT

A method for selecting items, the method comprising the steps of measuring (21) a pre-stimulus level of a physiological parameter of a user, selecting (22) an item based on a user profile and the pre-stimulus level of the physiological parameter, measuring (23) a post-stimulus level of the physiological parameter, determining (24) a stimulus effect by calculating a difference between the post-stimulus level and the pre-stimulus level, correcting (25) the stimulus effect using a model of an effect of the pre-stimulus level on the physiological parameter and updating (26) the user profile, using the corrected stimulus effect. The method may, e.g., be used for selecting media items in a music player or digital TV.

### FIELD OF THE INVENTION

This invention relates to a method for selecting items, the method comprising the step of measuring a level of a physiological parameter of a user. This invention further relates to a system for content item selection and to a computer program product for performing the method.

### BACKGROUND OF THE INVENTION

In, e.g., music players it is known to take into account an emotional or physiological state of the user when selecting music. Some music players use moodbased item selection. In a more simple form, a user may inform the music player of his or hers current mood. The mood based user profile then indicates for each song whether it is suitable for being played when the user is in said current mood. The music player may then, based on the mood information in the mood based profile, select the most suitable song or may increase or decrease a probability of an item being selected in a random mode. A more advanced music player may comprise means for measuring a physiological parameter of the user, the physiological parameter being, more or less, representative of the mood of the user. Such a music player may determine a user's mood without requiring user input. Some physiological states which may be relevant for determining a mood of a user are heart rate, skin temperature and skin conductance level. When determining the physiological parameter before and after the selection of a song, the effect of the selected song on the physiological parameter may be determined. This effect may be used for refining the content of the mood based user profile, in such a way that future song selections may better suit the current mood of the user.

One of the problems with determining physiological parameters is the fact that the physiological signals are inherently noisy. Beside the mental state of the user, there are many factors that influence the physiological signals. The physiological signals may also be influenced by activity of the user or varying environmental conditions. For example, standing up and walking away may increase heart rate and exposure to sunlight may increase skin temperature. The noisy character of the physiological signals makes it problematic to infer a user's mental state therefrom.

### SUMMARY OF THE INVENTION

It is desirable to provide a method for mood based item selection, capable of measuring physiological signals with reduced influence of noise.

This is achieved by providing a method for selecting items, the method comprising steps of measuring a pre-stimulus level of a physiological parameter of a user, selecting an item based on a user profile and the pre-stimulus level of the physiological parameter, measuring a post-stimulus level of the physiological parameter, determining a stimulus effect by calculating a difference between the post-stimulus level and the pre-stimulus level, correcting the stimulus effect using a model of an effect of the pre-stimulus level on the physiological parameter and updating the user profile, using the corrected stimulus effect.

The inventors have realized that the noise in the physiological signals is for a large part caused by the tendency of these signals to move towards a stable state. In statistics, this tendency is known as ‘regression to the mean’. For a user having, e.g., an exceptional high heart rate, there is only little chance that the next item to be selected will further increase the heart rate. For a user with, e.g., a very low skin temperature, there is a good chance of a skin temperature increase following the selection of an item. In such exceptional circumstances, the measured effect of an item on the measured physiological parameters may not be representative for the effect of this item on the user in other occasions. The inventors have not only realized that this regression to the mean for the physiological parameters is an important cause of the perceived noise, but they also found a way of compensating for this effect. For this purpose a model is used of an effect of the pre-stimulus level on the physiological parameter. The model predicts the effect of the regression to the mean for a given pre-stimulus level. This predicted regression effect is then used for correcting the stimulus effect (the difference between the post-stimulus level and the pre-stimulus level).

The regression model for a specific physiological model may be a general model, which is applicable to all users. However, in a preferred embodiment, the model used for correcting the stimulus effect is user dependent, which makes it even more accurate.

The method according to the invention may further comprise a step of determining a target physiological state for the user based on the pre-stimulus level of the physiological parameter, while the step of selecting is further based on the target physiological state and an expected stimulus effect of the selected item. The user profile and the regression model may be used for predicting the stimulus effect of an item. An item may be selected when the corresponding predicted stimulus effect and the pre-stimulus level are expected to bring the physiological value to the target state.

The method according to the invention may be used for selecting, e.g., an item from a plurality of songs, TV programs, pictures or lighting schemes. Also the selection of e.g. a sound level, light color, light intensity, or other actuator settings may be considered selection of an item. The method according to the invention thus is not limited to selecting media items.

According to a second aspect of the invention, a computer program product is provided for performing the above described method.

According to a third aspect of the invention a system is provided for performing the method according to the invention. The system comprises means for measuring a level of a physiological parameter of a user, a storage means for storing the user profile and a processor being operative to perform the method according to the invention.

These and other aspects of the invention are apparent from and will be elucidated with reference to the embodiments described hereinafter.

### BRIEF DESCRIPTION OF THE DRAWINGS

In the drawings:

FIG. 1 schematically shows a music player according to the invention,

FIG. 2 shows a flow diagram of a method according the invention,

FIG. 3 shows a graph for visualizing the regression model used in the method according to the invention.

### DETAILED DESCRIPTION OF THE INVENTION

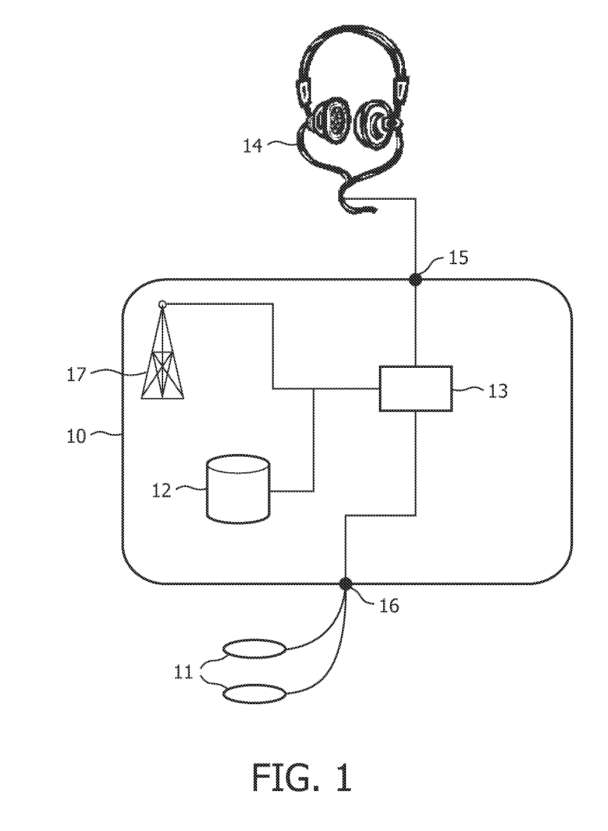

FIG. 1 schematically shows a music player 10 according to the invention. The music player 10 is only shown as an example. The invention potentially has many further applications. For example, the items to be selected may be TV programs for a digital TV, photos or other images in a photo album or video games in a gaming device. Another interesting application of the invention may be a an interactive lighting system in which lighting schemes are selected or generated in dependence of physiological parameters. For example, the intensity, color and direction of one or more light sources may be determined in dependence on measured physiological parameters. The effect of an applied lighting scheme on one or more users may be measured and stored in a user profile or users profile. Predictions of changes applied to the lighting system may be used for determining how to adapt the lighting scheme. Other applications of the method according to the invention are also possible.

The music player 10 described below uses a mood based user profile for selecting music or other audio content which is suitable for a user in a specific mood. The music player 10 further, indirectly via measuring the physiological parameters, determines the effect of an item on the user's mood. The music player 10 is also adapted to determine a target mood, make predictions of the effect of an item on the user's mood and select an item accordingly. Alternative music players or other devices applying the invention don't necessarily determine moods. For example, a music player used while running may measure heart rate and select music accordingly. In such a music player, the physiological parameter itself is controlled and mood doesn't play any role.

The music player 10 in FIG. 1 comprises a storage means 12 for storage of a user profile and a collection of audio tracks. Alternatively, the selectable audio may be received from an external source, such as a radio station. For this purpose, a receiver 17 may be comprised in the music player 10. When selecting a radio station, metadata sent together with the audio content may define a genre or the content of the radio program. The user profile would then be compared to the metadata for making a proper selection. Selection of audio content to be played is performed by the processor 13, which is coupled to the storage means 12 and an output 15 of the music player 10. A sound producing unit, such as headphones 14, or a speaker system may be coupled to the output 15 in order to make the selected audio content audible. The output 15 may alternatively be coupled to an amplifier or other sound processing equipment.

One or more sensors 11 are coupled to an input 16 of the music player 10 for measuring physiological parameters of the user. The parameters measured by the sensors 11 are representative of the mood of the user. Exemplary parameters to be measured are heart rate, skin temperature and skin conductance level. For these parameters, there is a known relation between mood and parameter level. Some ranges for a specific physiological parameter may correspond to specific moods. When multiple physiological parameters are measured, combinations of parameter values may correspond to specific moods. These known relations are used by the processor 13 for determining a mood of the user. These relations may also be used for determining which physiological parameter(s) should be controlled for realizing a transition to a different mood.

It is to be noted that in addition to physiological parameters, also further information may be used for determining a user's mood. For example, skin temperature may not only depend on mood, but also on environmental temperature. Environmental temperature and other external factors may thus be used for defining the relations between physiological parameters and mood.

The mood based user profile provides information about which audio tracks may be appreciated by the user, when he/she is in a specific mood. The processor 13 may thus use the mood based user profile to select audio tracks which are suitable for the current mood of the user. It is to be noted that the user may like entirely different music for different moods. For example, the user may like up-tempo party music when he is very excited, but not when he is sad.

In order to make valuable decisions about what audio content to select, the processor 13 is preferably arranged to calculate an expected effect of the audio track on the user's mood or on the related physiological parameters. Depending on the current mood, the processor 13 may determine a target mood. The target mood may also be influenced or determined by other factors, such as time of day, day of the week, weather, etc. An expected effect may not be very accurate, if the models used for calculating the expected effects are not compared to actual effects of the selected audio on the user. It is thus preferable to measure the actual effect of selecting an audio track, instead of only calculating an expected effect. For this purpose, a pre-stimulus level (before selection of the item) and a post-stimulus level (after selection of the item) of the physiological value(s) are measured. As will be elucidated below, these levels are used for accurately determining the effect of the selection of an item and the mood based user profile is updated accordingly.

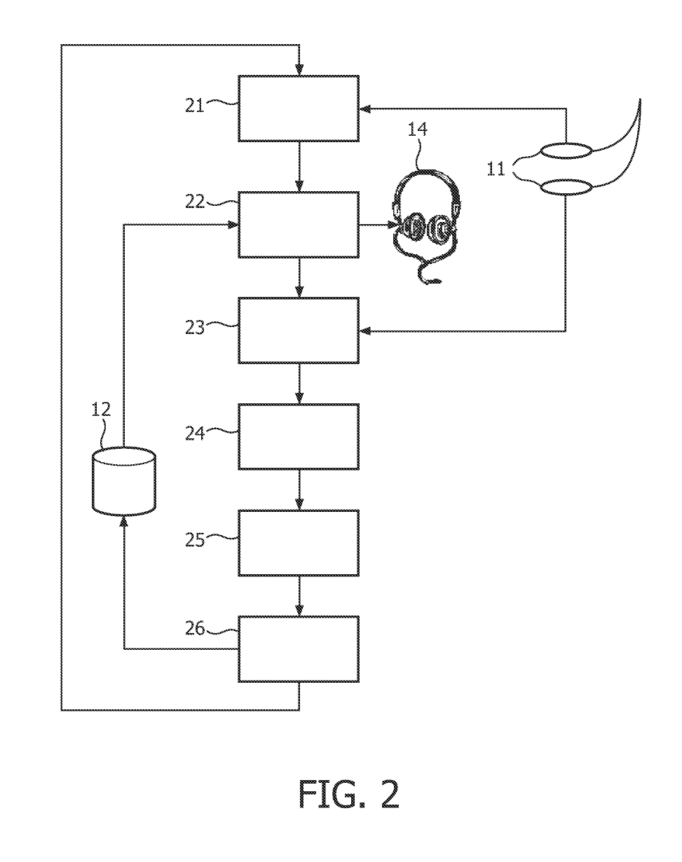

FIG. 2 shows a flow diagram of a method according the invention. In initial measurement step 21, the sensors 11 are used for measuring a pre-stimulus level of at least one physiological parameter of the user, e.g. heart rate. In the following selection step 22, an audio track or radio station is selected, based on information from the mood based user profile, stored in the storage means 12. The selected audio content is provided at the output 15 and, e.g., reproduced by the earphones 14 which are coupled to the output. In alternative devices other items may be selected. The method shown in FIG. 2 is also suitable for selection of, e.g., TV programs, pictures, or lighting schemes.

In order to make valuable decisions about what audio content to select, the processor 13 is preferably arranged to calculate an expected effect of the audio track on the user's mood or on the related physiological parameters. Depending on the current mood, the processor 13 may determine a target mood. The target mood may also be influenced or determined by other factors, such as time of day, day of the week, weather, etc.

The expected effect of an audio track on the physiological parameters depends on track specific information, stored in the storage means 12. The inventors have realized that also the current levels of the physiological parameters may play an important role in the calculation of the expected effect. Below, with reference to step 25 it will be elucidated how the current physiological parameter values may influence the expected effect. In selection step 22, the processor 13 may select the audio track that brings the user as close as possible to the target mood. Preferably, however, an audio track is randomly selected from a group of audio tracks having an expected beneficial effect. When randomly selecting an audio track, the processor 13 may use the expected effect to assign a probability to each track being available for selection. Using a partly random process for selecting audio, ensures that there is enough variation in what is being selected.

After selection step 22, a post-stimulus level of the physiological parameter is measured in further measurement step 23. This measurement is performed while or soon after the audio is being played. The post-stimulus level of the physiological parameter is measured using the sensors 11. For example, the post-stimulus level is measured a predetermined amount of time before the end of the selected audio. Preferably, the post-stimulus level is not measured directly after selection of the audio, because the selected audio first needs some time to affect the mood (and physiological parameters) of the user.

If the selected audio track induces a mood change for the user, then the level of the post-stimulus level differs from the pre-stimulus level. In effect determining step 24, the pre-stimulus level is compared to the post-stimulus level to determine the stimulus effect of the selected audio. The result of this comparison is however susceptible to noise. The audio track is not the only factor which may influence the measured physiological parameter. For example, personal activity or changing environmental conditions may also change the physiological parameter.

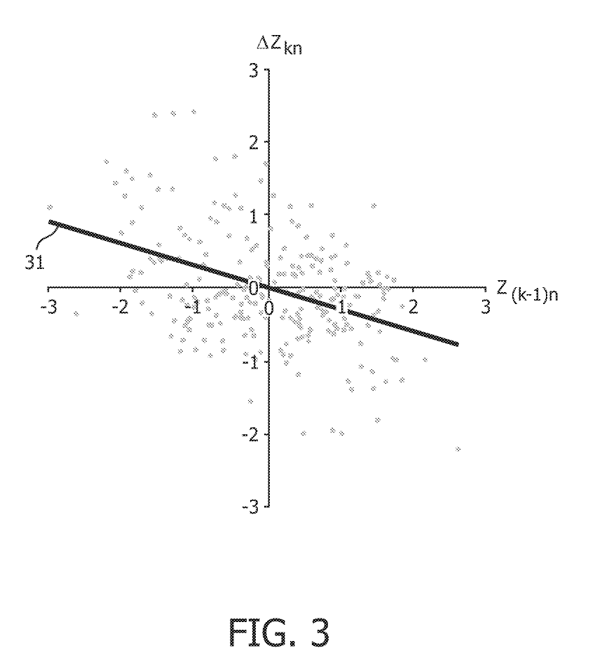

According to the invention, correction step 25 reduces the effect of noise on the attempts to determine the effect of selected audio on the measured physiological parameters. For the noise reduction in correction step 25, a model is used of an effect of the pre-stimulus level on the physiological parameter. The noise in the physiological signals is for a large part caused by the tendency of these signals to move towards a stable state. In statistics, this tendency is known as ‘regression to the mean’. For a user in an exceptional positive mood, there is only little chance that the next item to be selected will improve the mood. For a user in an exceptional negative mood, there is a good chance of mood improvement following the selection of an item. In such exceptional circumstances, the measured effect of an item on the measured physiological parameters may not be representative for the effect of this item on the user in other occasions. The used model (also called regression model) predicts the effect of the regression to the mean for a given pre-stimulus level. This predicted regression effect is then used for correcting the stimulus effect. The regression model is further explained below, with reference to FIG. 3.

In update step 26, the corrected stimulus effect is used for updating the mood based user profile. Every time an audio track is selected, new measurements are used for determining the corrected stimulus effect of that audio track on the user. The more often an audio track is selected, the more accurate the information about the effect of that audio track will be. The corrected stimulus effect information in the mood profile data base is used for making valuable selections in selection step 22. In this way, a closed-loop system is established. A user profile and physiological measurements are used for determining a target state and selecting a suitable item or other actuator setting. The effect of the selected item is measured and a new item may be selected. The effect of an audio track may change over time. A song making the user very happy in one year may have a less positive effect or even a negative effect on the user's mood a few years later. When updating the user profile, more recent information may be considered more important than older information.

FIG. 3 shows a graph for visualizing the regression model used in the method according to the invention. In FIG. 3, the following is to be seen. FIG. 3 plots the effect of a song k in measurement session n on a physiological parameter. For example, during the last minute of each song the parameter is measured and the mean value over this last minute is denoted by xₖₙ. The physiological parameter has a mean value, μₙ, and a standard deviation, σₙ, over the whole measurement session n. Along the horizontal axis, a standardized parameter value, zₖₙ, is plotted, given by formula (1).

zₖₙ=(xₖₙ−μₙ)/σₙ (1)

Along the vertical axis, delta scores, Δₖₙ, are plotted, indicating the effect of the song k on the physiological parameter.

Δzₖₙ=zₖₙ−z₍ₖ₋₁₎ₙ,k>1 (2)

The dots in the figure represent measured parameter levels. The line 31 depicts the regression line, representing the regression model to be used in correcting step 25:

yₖₙ=w₁z₍ₖ₋₁₎ₙ+w₀, (3)

wherein w₀ and w₁ are the parameters of the regression line 31. When w₀ and w₁ are assessed, the corrected stimulus effects Δ′zₖₙ are computed by subtracting the value of regression line yₖₙ at z₍ₖ₋₁₎ₙ from the delta scores Δₖₙ:

Δ′zₖₙ=Δzₖₙ−yₖₙ (4)

The regression line 31 may differ from person to person. It is thus preferable to estimate this relation for every user separately. Each measurement of a physiological parameter may be stored in the user profile and may be used for refining the regression model. The device using the regression model may be sold with a predetermined regression model which may be update over time with information derived during use of the device. The dots in FIG. 3, may thus be part of the sold device, or may be determined during use. The regression line 31 may also differ for different physiological parameters. So, also different regression lines 31 may be used for different physiological parameters. It is to be noted that above, a fairly simple linear regression model is described. However, the regression model may take a more complicated form. It will be appreciated that the invention also extends to computer programs, particularly computer programs on or in a carrier, adapted for putting the invention into practice. The program may be in the form of source code, object code, a code intermediate source and object code such as partially compiled form, or in any other form suitable for use in the implementation of the method according to the invention. It will also be appreciated that such a program may have many different architectural designs. For example, a program code implementing the functionality of the method or system according to the invention may be subdivided into one or more subroutines. Many different ways to distribute the functionality among these subroutines will be apparent to the skilled person. The subroutines may be stored together in one executable file to form a self-contained program. Such an executable file may comprise computer executable instructions, for example processor instructions and/or interpreter instructions (e.g. Java interpreter instructions). Alternatively, one or more or all of the subroutines may be stored in at least one external library file and linked with a main program either statically or dynamically, e.g. at run-time. The main program contains at least one call to at least one of the subroutines. Also, the subroutines may comprise function calls to each other. An embodiment relating to a computer program product comprises computer executable instructions corresponding to each of the processing steps of at least one of the methods set forth. These instructions may be subdivided into subroutines and/or be stored in one or more files that may be linked statically or dynamically. Another embodiment relating to a computer program product comprises computer executable instructions corresponding to each of the means of at least one of the systems and/or products set forth. These instructions may be subdivided into subroutines and/or be stored in one or more files that may be linked statically or dynamically.

The carrier of a computer program may be any entity or device capable of carrying the program. For example, the carrier may include a storage medium, such as a ROM, for example a CD ROM or a semiconductor ROM, or a magnetic recording medium, for example a floppy disc or hard disk. Further the carrier may be a transmissible carrier such as an electrical or optical signal, which may be conveyed via electrical or optical cable or by radio or other means. When the program is embodied in such a signal, the carrier may be constituted by such cable or other device or means. Alternatively, the carrier may be an integrated circuit in which the program is embedded, the integrated circuit being adapted for performing, or for use in the performance of, the relevant method.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A method for selecting items, the method comprising the steps of:
measuring (21) a pre-stimulus level of a physiological parameter of a user,
selecting (22) an item based on a user profile and the pre-stimulus level of the physiological parameter,
measuring (23) a post-stimulus level of the physiological parameter,
determining (24) a stimulus effect by calculating a difference between the post-stimulus level and the pre-stimulus level,
correcting (25) the stimulus effect using a model of an effect of the pre-stimulus level on the physiological parameter, and
updating (26) the user profile, using the corrected stimulus effect.

2. The method for selecting items as claimed in claim 1, wherein the model is user dependent.

3. The method for selecting items as claimed in claim 1, further comprising a step of determining a target physiological state for the user based on the pre-stimulus level of the physiological parameter, and wherein the step of selecting is further based on the target physiological state and an expected stimulus effect of the selected item, the expected stimulus effect being based on the user profile.

4. The method for selecting items as claimed in claim 1, wherein the item is selected from a plurality of songs, TV programs, pictures or lighting schemes.

5. The method for selecting items as claimed in claim 1, wherein the step of selecting (22) is further based on a pre-stimulus level of at least one further physiological parameter of the user, the method further comprising the steps of:
measuring (21) a pre-stimulus level of the at least one further physiological,
measuring (23) a post-stimulus level of the at least one further physiological parameter,
determining (24) a further stimulus effect by calculating a difference between the post-stimulus level and the pre-stimulus level of the at least one further physiological level,
correcting (25) the further stimulus effect using a model of an effect of the pre-stimulus level on the at least one further physiological parameter,
updating (26) the user profile, using the corrected further stimulus effect.

6. A computer program product, which program is operative to cause a processor to perform a method as claimed in claim 1.

7. A system (10) for selecting items, the system (10) comprising:
means (11) for measuring a level of a physiological parameter of a user,
a storage means (12) for storing a user profile,
a processor (13) being operative to:
measure a pre-stimulus level of the physiological parameter,
select an item based on the user profile and the pre-stimulus level of the physiological parameter,
measure a post-stimulus level of the physiological parameter,
determine a stimulus effect by calculating a difference between the post-stimulus level and the pre-stimulus level,
correct the stimulus effect using a model of an effect of the pre-stimulus level on the physiological parameter, and to
update the user profile, using the corrected stimulus effect.
